(19) United States
(12) Patent Application Publication
(10) Pub. No.: US 2021/0030276 A1
(43) Pub. Date: Feb. 4, 2021

(54) Remote Health Monitoring Systems and Method

(71) Applicant: DawnLight Technologies Inc., Palo Alto, CA (US)

(72) Inventors: Jia Li, Palo Alto, CA (US); Fan Li, Palo Alto, CA (US); Nan Liu, Saratoga, CA (US); Christopher Wing, San Francisco, CA (US); Ke Zhai, Santa Clara, CA (US); Ning Zhang, Mountain View, CA (US); Erheng Zhong, Fremont, CA (US)

(21) Appl. No.: 16/524,772

(22) Filed: Jul. 29, 2019

(51) Int. Cl. A61B 5/00; A61B 5/0205; A61B 5/0255; A61B 5/103; G06N 3/02; G06N 20/20; A61B 7/02

(52) U.S. Cl. CPC A61B 5/0022; A61B 5/0205; A61B 5/0255; A61B 5/1032; A61B 5/0816; A61B 5/4815; G06N 3/02; G06N 20/20; A61B 7/026; A61B 5/7267

(57) ABSTRACT

Embodiments of remote health monitoring systems and methods are disclosed. In one embodiment, a plurality of sensors is configured for contact-free monitoring of at least one bodily function. A signal processing module communicatively coupled with the plurality of sensors is configured to receive data from the plurality of sensors. A first sensor is configured to generate a first set of data associated with a first bodily function. A second sensor is configured to generate a second set of data associated with a second bodily function. A third sensor is configured to generate a third set of data associated with a third bodily function. The signal processing module is configured to receive and process the first set of data, the second set of data, and the third set of data. The signal processing module is configured to generate at least one diagnosis of a health condition responsive to the processing.

### BACKGROUND

### Technical Field

The present disclosure relates to systems and methods that perform non-contact health monitoring of an individual using different sensing modalities and associated signal processing techniques that include machine learning.

### Background Art

Currently, methods employed to monitor pulmonary and respiratory diseases such as chronic obstructive pulmonary disease (COPD), asthma, obstructive sleep apnea (OSA), and other conditions such as congestive heart failure (CHF) involve sensors attached to a patient's body. For example, a pulmonary test function requires a patient to wear a mask that increases a probability of patient discomfort and associated noncompliance with the monitoring method. Polysomnography (PSG) for OSA requires an overnight hospital stay while a patient is physically connected to 10-15 channels of measurement. This turns out to be inconvenient and expensive. There exists a need for a non-contact (i.e., contact-free) method of monitoring and diagnosing pulmonary and respiratory diseases such as COPD, asthma, OSA, and conditions such as CHF, without significantly introducing patient discomfort or requiring a hospital visit.

### SUMMARY

Embodiments of apparatuses configured to perform a contact-free detection of one or more health conditions may include: a plurality of sensors configured for contact-free monitoring of at least one bodily function; and a signal processing module communicatively coupled with the plurality of sensors; wherein the signal processing module is configured to receive data from the plurality of sensors; wherein a first sensor of the plurality of sensors is configured to generate a first set of quantitative data associated with a first bodily function; wherein a second sensor of the plurality of sensors is configured to generate a second set of quantitative data associated with a second bodily function; wherein a third sensor of the plurality of sensors is configured to generate a third set of quantitative data associated with a third bodily function; wherein the signal processing module is configured to process the first set of quantitative data, the second set of quantitative data, and the third set of quantitative data, and wherein the signal processing module is configured to process at least one of the sets of quantitative data using a machine learning module; and wherein the signal processing module is configured to generate, responsive to the processing, at least one diagnosis of a health condition.

Embodiments of apparatuses configured to perform a contact-free detection of one or more health conditions may include one or all or any of the following:

The first bodily function may be one of heartbeat and respiration, the second bodily function may be a daily activity, and the third bodily function may be coughing, snoring, expectoration and/or wheezing.

The first sensor may be a radar, the second sensor may be a visual sensor, and the third sensor may be an audio sensor.

The radar may be a millimeter wave radar, the visual sensor may be a depth sensor or an RGB sensor, and the audio sensor may be a microphone.

The radar may be configured to generate quantitative data associated with heartbeat and/or breathing, the visual sensor may be configured to generate quantitative data associated with a daily activity, and the audio sensor may be configured to generate quantitative data associated with coughing, snoring, wheezing and/or expectoration.

Data generated using the audio sensor may be processed using a combination of a Mel-frequency Cepstrum and a deep learning model associated with the machine learning module.

Data generated using the radar may be processed using static clutter removal, band pass filtering, time-frequency analysis, wavelet transforms, spectrograms, and/or a deep learning model associated with the machine learning module.

The health condition may be a respiratory health condition.

The respiratory health condition may be one of OSA, COPD, and asthma.

Results from processing the first set of quantitative data, the second set of quantitative data, and the third set of quantitative data may be combined to generate the diagnosis.

Embodiments of methods for performing a contact-free detection of one or more health conditions may include: generating, using a first sensor of a plurality of sensors, a first set of quantitative data associated with a first bodily function of a body, wherein the first sensor does not contact the body; generating, using a second sensor of the plurality of sensors, a second set of quantitative data associated with a second bodily function of the body, wherein the second sensor does not contact the body; generating, using a third sensor of the plurality of sensors, a third set of quantitative data associated with a third bodily function of the body, wherein the third sensor does not contact the body; processing, using a signal processing module, the first set of quantitative data, the second set of quantitative data, and the third set of quantitative data, wherein the signal processing module is communicatively coupled with the plurality of sensors, and wherein at least one of the first set of quantitative data, the second set of quantitative data, and the third set of quantitative data is processed using a machine learning module; and generating, using the signal processing module, responsive to the processing, at least one diagnosis of a health condition.

Embodiments of methods for performing a contact-free detection of one or more health conditions may include one or more or all of the following:

The first bodily function may be heartbeat and/or respiration, the second bodily function may be a daily activity, and the third bodily function may be coughing, snoring, sneezing, expectoration and/or wheezing.

The first sensor may be a radar, the second sensor may be a visual sensor, and the third sensor may be an audio sensor.

The radar may be a millimeter wave radar, the visual sensor may be a depth sensor or an RGB sensor, and the audio sensor may be a microphone.

The method may further include: generating, using the radar, quantitative data associated with heartbeat and/or respiration; generating, using the visual sensor, quantitative data associated with a daily activity; and generating, using the audio sensor, quantitative data associated with coughing, snoring, sneezing, wheezing and/or expectoration.

The method may further include receiving, by the signal processing module, the first set of quantitative data associated with an RF signal generated using the radar; subtracting, using the signal processing module, a moving average associated with the first set of quantitative data; band-pass filtering, using the signal processing module, the first set of quantitative data; performing, using the signal processing module, time-frequency analysis on the first set of quantitative data using wavelet transforms; and predicting, using the signal processing module, a user heart rate and a user respiratory rate using a deep learning model and a spectrogram function.

The method may further include receiving, using the signal processing module, the third set of quantitative data associated with an audio signal from the audio sensor; producing, using the signal processing module, a Mel-frequency cepstrum using time-frequency analysis performed on the third set of quantitative data; and determining, using the signal processing module, a presence of a cough, a snore and/or a wheeze associated with a user.

The health condition may be a respiratory health condition.

The respiratory health condition may be OSA, COPD, and/or asthma.

Results from processing the first set of quantitative data, the second set of quantitative data, and the third set of quantitative data may be combined to generate the diagnosis.

### BRIEF DESCRIPTION OF THE DRAWINGS

Non-limiting and non-exhaustive embodiments of the present disclosure are described with reference to the following figures, wherein like reference numerals refer to like parts throughout the various figures unless otherwise specified.

FIG. 1 is a block diagram depicting an embodiment of a remote health monitoring system implementation.

FIG. 2 is a block diagram depicting an embodiment of a signal processing module that is configured to implement certain functions of a remote health monitoring system.

FIG. 3 is a block diagram depicting an embodiment of a diagnosis module.

FIG. 4 is a schematic diagram depicting a heatmap.

FIG. 5 is a block diagram depicting an embodiment of a system architecture of a remote health monitoring system.

FIG. 6 is a flow diagram depicting an embodiment of a method to generate a diagnosis of a health condition.

FIG. 7 is a flow diagram depicting an embodiment of a method to predict a user heart rate and a user respiratory rate.

FIG. 8 is a flow diagram depicting an embodiment of a method to determine a presence of a cough, a snore, or a wheeze.

FIG. 9 is a schematic diagram depicting a processing flow of multiple heatmaps using neural networks.

FIG. 10 is a block diagram depicting an embodiment of a system architecture of a remote health monitoring system.

### DETAILED DESCRIPTION

In the following description, reference is made to the accompanying drawings that form a part thereof, and in which is shown by way of illustration specific exemplary embodiments in which the disclosure may be practiced. These embodiments are described in sufficient detail to enable those skilled in the art to practice the concepts disclosed herein, and it is to be understood that modifications to the various disclosed embodiments may be made, and other embodiments may be utilized, without departing from the scope of the present disclosure. The following detailed description is, therefore, not to be taken in a limiting sense.

Reference throughout this specification to “one embodiment,” “an embodiment,” “one example,” or “an example” means that a particular feature, structure, or characteristic described in connection with the embodiment or example is included in at least one embodiment of the present disclosure. Thus, appearances of the phrases “in one embodiment,” “in an embodiment,” “one example,” or “an example” in various places throughout this specification are not necessarily all referring to the same embodiment or example. Furthermore, the particular features, structures, databases, or characteristics may be combined in any suitable combinations and/or sub-combinations in one or more embodiments or examples. In addition, it should be appreciated that the figures provided herewith are for explanation purposes to persons ordinarily skilled in the art and that the drawings are not necessarily drawn to scale.

Embodiments in accordance with the present disclosure may be embodied as an apparatus, method, or computer program product. Accordingly, the present disclosure may take the form of an entirely hardware-comprised embodiment, an entirely software-comprised embodiment (including firmware, resident software, micro-code, etc.), or an embodiment combining software and hardware aspects that may all generally be referred to herein as a “circuit,” “module,” or “system.” Furthermore, embodiments of the present disclosure may take the form of a computer program product embodied in any tangible medium of expression having computer-usable program code embodied in the medium.

Any combination of one or more computer-usable or computer-readable media may be utilized. For example, a computer-readable medium may include one or more of a portable computer diskette, a hard disk, a random access memory (RAM) device, a read-only memory (ROM) device, an erasable programmable read-only memory (EPROM or Flash memory) device, a portable compact disc read-only memory (CDROM), an optical storage device, a magnetic storage device, and any other storage medium now known or hereafter discovered. Computer program code for carrying out operations of the present disclosure may be written in any combination of one or more programming languages. Such code may be compiled from source code to computer-readable assembly language or machine code suitable for the device or computer on which the code will be executed.

Embodiments may also be implemented in cloud computing environments. In this description and the following claims, “cloud computing” may be defined as a model for enabling ubiquitous, convenient, on-demand network access to a shared pool of configurable computing resources (e.g., networks, servers, storage, applications, and services) that can be rapidly provisioned via virtualization and released with minimal management effort or service provider interaction and then scaled accordingly. A cloud model can be composed of various characteristics (e.g., on-demand self-service, broad network access, resource pooling, rapid elasticity, and measured service), service models (e.g., Software as a Service (“SaaS”), Platform as a Service (“PaaS”), and Infrastructure as a Service (“IaaS”)), and deployment models (e.g., private cloud, community cloud, public cloud, and hybrid cloud).

The flow diagrams and block diagrams in the attached figures illustrate the architecture, functionality, and operation of possible implementations of systems, methods, and computer program products according to various embodiments of the present disclosure. In this regard, each block in the flow diagrams or block diagrams may represent a module, segment, or portion of code, which includes one or more executable instructions for implementing the specified logical function(s). It will also be noted that each block of the block diagrams and/or flow diagrams, and combinations of blocks in the block diagrams and/or flow diagrams, may be implemented by special purpose hardware-based systems that perform the specified functions or acts, or combinations of special purpose hardware and computer instructions. These computer program instructions may also be stored in a computer-readable medium that can direct a computer or other programmable data processing apparatus to function in a particular manner, such that the instructions stored in the computer-readable medium produce an article of manufacture including instruction means which implement the function/act specified in the flow diagram and/or block diagram block or blocks.

The systems and methods described herein relate to a remote health monitoring system that is configured to perform remote and contact-free monitoring and diagnosis of one or more health conditions associated with a patient. In some embodiments, the health conditions include respiratory health conditions such as COPD, CHF, asthma, and OSA. In other embodiments, health conditions such as CHF may be monitored and diagnosed by the remote health monitoring system. Some embodiments of the remote health monitoring system use multiple sensors with associated signal processing and machine learning to perform the diagnoses, as described herein.

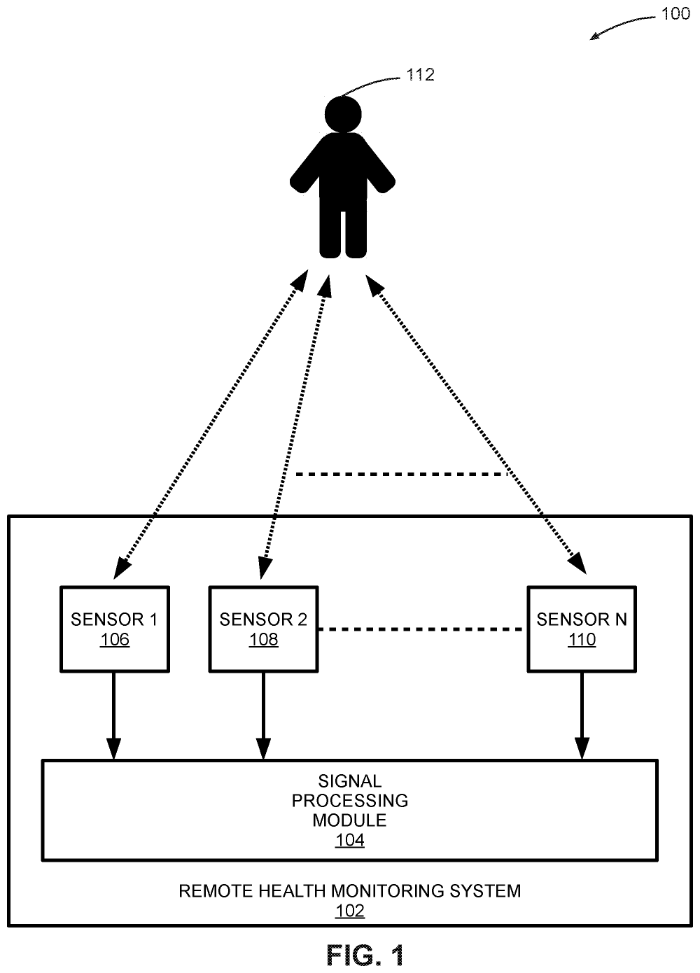

FIG. 1 is a block diagram depicting an embodiment of a remote health monitoring system implementation 100. In some embodiments, remote health monitoring implementation 100 includes a remote health monitoring system 102 that is configured to monitor and diagnose one or more health conditions associated with a user 112. In particular embodiments, remote health monitoring system 102 is configured to generate at least one diagnosis of a health condition, using a sensor 1 106, a sensor 2 108, through a sensor N 110 included in remote health monitoring system 102. In some embodiments, remote health monitoring system 102 includes a signal processing module 104 that is communicatively coupled to each of sensor 1 106 through sensor N 110, where signal processing module 104 is configured to receive data generated by each of sensor 1 106, through sensor N 110.

In some embodiments, each of sensor 1 106 through sensor N 110 is configured to remotely measure and generate data associated with a bodily function of user 112, in a contact-free manner. For example, sensor 1 106 may be configured to generate a first set of quantitative data associated with a measurement of a first bodily function such as a heartbeat, a breathing process or a respiration process; sensor 2 108 may be configured to generate a second set of quantitative data associated with a measurement of a second bodily function such as an activity of daily life (also referred to as a “daily activity,” or “ADL”); and sensor N 110 may be configured to generate a third set of quantitative data associated with a measurement of a third bodily function such as a cough, a snore, an expectoration, or a wheeze. In some embodiments, an activity of daily life includes activities performed by user 112 that include sitting, standing, walking, getting up from a chair, eating, sleeping, laying down, and so on. Other sensors from a sensing group comprising sensor 1 106 through sensor N 110 may measure other bodily functions such as vital signs, and generate quantitative data associated with those bodily functions.

In some embodiments, signal processing module 104 is configured to process the first set of quantitative data, the second set of quantitative data, and the third set of quantitative data to generate at least one diagnosis of a health condition such as asthma, COPD, OSA, or CHF. Signal processing module 104 may also be configured to generate a notification or an alert of a health condition responsive to processing the multiple sets of quantitative data. In particular embodiments, signal processing module 104 may use a machine learning algorithm to process at least one of the sets of quantitative data, as described herein.

In some embodiments, data processed by signal processing module 104 may include current (or substantially real-time) data that is generated by sensor 1 106 through sensor N 110 at a current time instant. In other embodiments, data processed by signal processing module 104 may be historical data generated by sensor 1 106 through sensor N 110 at one or more earlier time instants. In still other embodiments, data processed by signal processing module 104 may be a combination of substantially real-time data and historical data.

In some embodiments, each of sensor 1 106 through sensor N 110 is a contact-free (or contactless, or non-contact) sensor, which implies that each of sensor 1 106 through sensor N 110 is configured to function with no physical contact or minimal physical contact with user 112. For example, sensor 1 106 may be a radar that is configured to remotely perform ranging and detection functions associated with a bodily function such as heartbeat or respiration; sensor 2 108 may be a visual sensor that is configured to remotely sense daily activities; sensor N 110 may be an audio sensor that is configured to remotely sense a cough, a snore, a wheeze or an expectoration. In some embodiments, the radar is a millimeter wave radar, the visual sensor is a depth sensor or a red-green-blue (RGB) sensor, and the audio sensor is a microphone. Operational details of example sensors that may be included in a group comprising sensor 1 106 through sensor N 110 are provided herein. Additionally, any of the sensors could be a combination of sensor types, for example the visual sensor could include a depth sensor and an RGB sensor, the audio sensor could include multiple audio inputs, and so forth.

Using non-contact sensing for implementing remote health monitoring system 102 provides several advantages. Non-contact sensors make an implementation of remote health monitoring system 102 non-intrusive and easy to set up in, for example, a home environment for long term continuous monitoring. Using a machine learning based sensor fusion approach produces accurate measurements without requiring expensive devices such as EEGs. Also, from a perspective of compliance with health standards, remote health monitoring system 102 requires minimal to no efforts on behalf of a patient (i.e., user 112) to install and operate the system; hence, such an embodiment of remote health monitoring system 102 would not violate any compliance regulations.

One example operation of remote health monitoring system 102 is based on the following steps:

Combining sets of quantitative data from the radar, the visual sensor, and the audio sensor to generate quantitative data sets associated with a heartbeat and respiratory activity (such as respiratory motion), actions from daily activities, and audio signals respectively.

Performing data processing and signal processing based on deep learning methods to produce metrics relevant to one or more diagnoses (e.g., heartbeat, respiration, cough, etc.).

Combining the metrics using machine-learned models to generate a diagnosis.

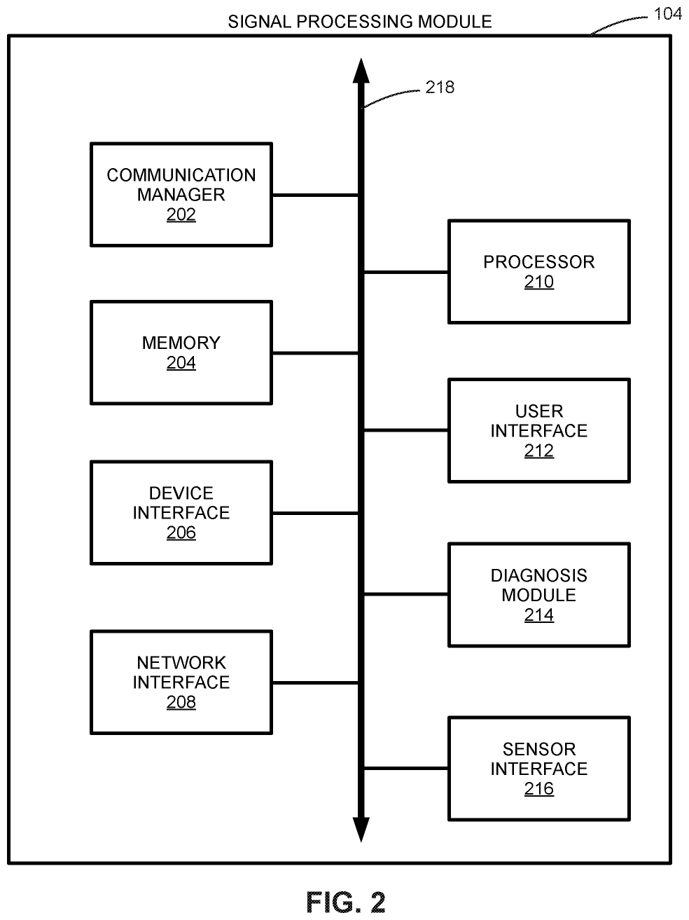

FIG. 2 is a block diagram depicting an embodiment of a signal processing module 104 that is configured to implement certain functions of a remote health monitoring system. In some embodiments, signal processing module 104 includes a communication manager 202, where communication manager 202 is configured to manage communication protocols and associated communication with external peripheral devices as well as communication within other components in signal processing module 104. For example, communication manager 202 may be responsible for generating and maintaining the interface between signal processing module 104 and sensor 1 106 through sensor N 110. Communication manager 202 may also be responsible for managing communication between the different components within signal processing module 104.

Some embodiments of signal processing module 104 include a memory 204 that may include both short-term memory and long-term memory. Memory 204 may be used to store, for example, substantially real-time and historical quantitative data sets generated by sensor 1 106 through sensor N 110. Memory 204 may be comprised of any combination of hard disk drives, flash memory, random access memory, read-only memory, solid state drives, and other memory components.

In some embodiments, signal processing module 104 includes a device interface 206 that is configured to interface signal processing module 104 with one or more external devices such as an external hard drive, an end user computing device (e.g., a laptop computer or a desktop computer), and so on. Device interface 206 generates the necessary hardware communication protocols associated with one or more communication protocols such as a serial peripheral interface (SPI), a serial interface, a parallel interface, a USB interface, and so on.

A network interface 208 included in some embodiments of signal processing module 104 includes any combination of components that enable wired and wireless networking to be implemented. Network interface 208 may include an Ethernet interface, a WiFi interface, and so on. In some embodiments, network interface 208 allows remote health monitoring system 102 to send and receive data over a local network or a public network.

Signal processing module 104 also includes a processor 210 configured to perform functions that may include generalized processing functions, arithmetic functions, and so on. Signal processing module 104 is configured to process one or more sets of quantitative data generated by sensor 1 106 through sensor N 110. Any artificial intelligence algorithms or machine learning algorithms (e.g., neural networks) associated with remote health monitoring system 102 may be implemented using processor 210.

In some embodiments, signal processing module 104 may also include a user interface 212, where user interface 212 may be configured to receive commands from user 112 (or another user, such as a health care worker, family member or friend of the user 112, etc.), or display information to user 112 (or another user). User interface 212 enables a user to interact with remote health monitoring system 102. In some embodiments, user interface 212 includes a display device to output data to a user; one or more input devices such as a keyboard, a mouse, a touchscreen, one or more push buttons, one or more switches; and other output devices such as buzzers, loudspeakers, alarms, LED lamps, and so on.

Some embodiments of signal processing module 104 include a diagnosis module 214 that is configured to process a plurality of sets of quantitative data generated by sensor 1 106 through sensor N 110 in conjunction with processor 210, and determine at least one diagnosis of a health condition associated with user 112. In some embodiments, diagnosis module 214 processes the plurality of sets of quantitative data using one or more machine learning algorithms such as neural networks, linear regression, a support vector machine, and so on. Details about diagnosis module 214 are presented herein.

In some embodiments, signal processing module 104 includes a sensor interface 216 that is configured to implement necessary communication protocols that allow signal processing module 104 to receive data from sensor 1 106, through sensor N 110.

A data bus 218 included in some embodiments of signal processing module 104 is configured to communicatively couple the components associated with signal processing module 104 as described above.

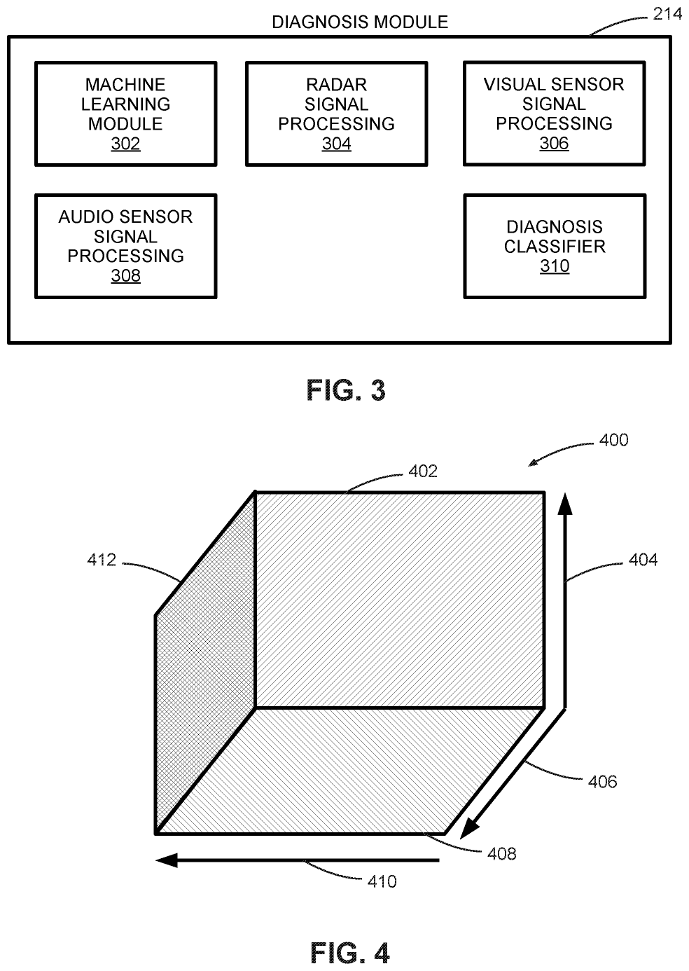

FIG. 3 is a block diagram depicting an embodiment of a diagnosis module 214. In some embodiments, diagnosis module 214 includes a machine learning module 302 that is configured to implement one or more machine learning algorithms that enable remote health monitoring system 102 to intelligently monitor and diagnose one or more health conditions associated with user 112. In some embodiments, machine learning module 302 is used to implement one or more machine learning structures such as a neural network, a linear regression, a support vector machine (SVM), or any other machine learning algorithm. In implementations, for large sets of quantitative data a neural network is a preferred algorithm in machine learning module 302.

In some embodiments, diagnosis module 214 includes a radar signal processing 304 that is configured to process a set of quantitative data generated by a radar sensor included in sensor 1 106 through sensor N 110. Diagnosis module 214 also includes a visual sensor signal processing 306 that is configured to process a set of quantitative data generated by a visual sensor included in sensor 1 106 through sensor N 110. Diagnosis module 214 also includes an audio sensor signal processing 308 that is configured to process a set of quantitative data generated by an audio sensor included in sensor 1 106 through sensor N 110.

In some embodiments, diagnosis module 214 includes a diagnosis classifier 310 that is configured to generate a diagnosis of at least one health condition associated with user 112, responsive to diagnosis module 214 processing one or more sets of quantitative data generated by sensor 1 106 through sensor N 110.

FIG. 4 is a schematic diagram depicting a heatmap 400. In some embodiments, heatmap 400 is generated responsive to signal processing module 104 processing a set of quantitative data generated by a radar. Details about the radar used in remote health monitoring system 102 are described herein. In particular embodiments, the set of quantitative data is processed by radar signal processing 304, where the radar is configured to generate quantitative data associated with RF signal reflections. In some embodiments, the radar is a millimeter wave frequency-modulated continuous wave radar (FMCW).

In some embodiments, heatmap 400 is generated based on a view 412 associated with the radar. View 412 is a representation of a view of an environment associated with user 112, where user 112 is included in a field of view of the radar. Responsive to processing RF reflection data associated with view 412, radar signal processing 304 generates a horizontal-depth heatmap 408 and a vertical-depth heatmap 402, where each of horizontal-depth heatmap 408 and vertical-depth heatmap 402 are referenced to a vertical axis 404, a horizontal axis 406, and a depth axis 410. In some embodiments, heatmap 400 is used as a basis for generating one or more sets of quantitative data associated with a heartbeat and a respiration of user 112.

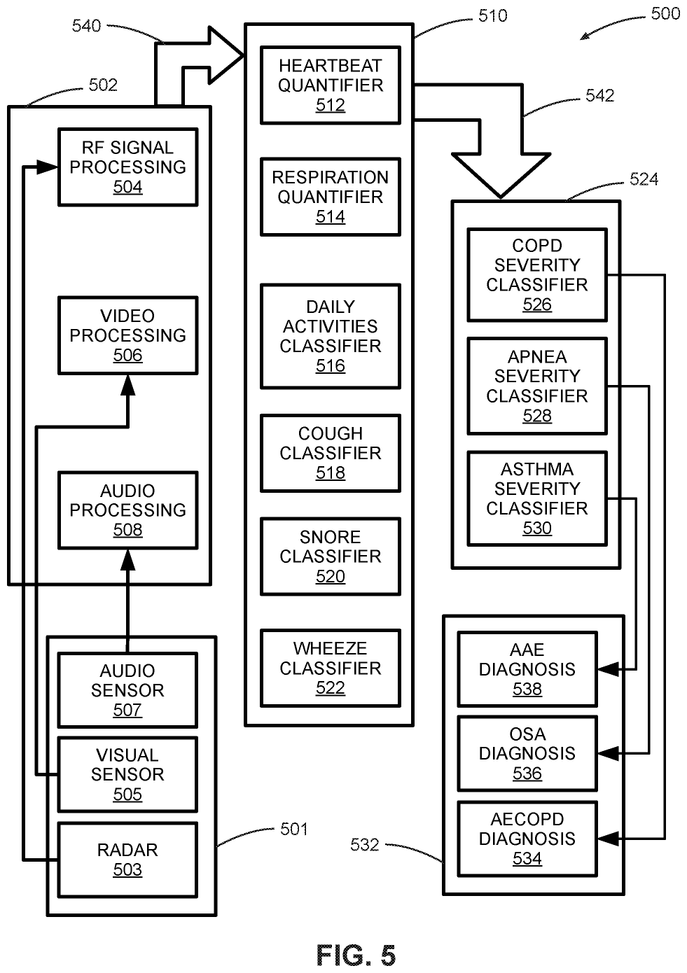

FIG. 5 is a block diagram depicting an embodiment of a system architecture 500 of a remote health monitoring system. In some embodiments, system architecture 500 includes a sensor layer 501. Sensor layer 501 includes a plurality of sensors configured to generate one or more sets of quantitative data associated with measuring one or more bodily functions associated with user 112. In some embodiments, sensor layer 501 includes sensor 1 106 through sensor N 110. In particular embodiments, sensor layer 501 includes a radar 503, a visual sensor 505, and an audio sensor 507.

In some embodiments, radar 503 is a millimeter wave frequency-modulated continuous wave radar that is designed for indoor use. Visual sensor 505 is configured to generate visual data associated with user 112. In some embodiments, visual sensor may include a depth sensor and/or an RGB sensor. Audio sensor 507 is configured to generate audio data associated with user 112.

In some embodiments, system architecture 500 includes a detection layer 502 that is configured to receive and process one or more sets of quantitative data generated by sensor layer 501. Detection layer 502 is configured to receive a set of quantitative data (also referred to herein as “sensor data”) from sensor layer 501. Detection layer 502 processes this sensor data to extract clinically-relevant signals from the sensor data. In particular embodiments, detection layer 502 includes an RF signal processing 504 that is configured to receive sensor data from radar 503, a video processing 506 that is configured to receive sensor data from visual sensor 505, and an audio processing 508 that is configured to receive sensor data from audio sensor 507.

In some embodiments, radar 503 is a millimeter wave frequency-modulated continuous wave radar. Radar 503 is capable of capturing fine motions of user 112 that include breathing and heartbeat. Signals associated with breathing and heartbeat are important signals for measuring cardiopulmonary functions. In particular embodiments, sensor data generated by radar 503 is processed by RF signal processing 504 to generate a heatmap such as heatmap 400. In embodiments, processing data generated by radar 503 involves the following steps performed by RF signal processing 504:

Static clutter removal: Processing data generated by radar 503 involves background modeling and removal. In this setup, the background clutters are mostly static and can be easily detected and removed using, for example, a moving average. Post-clutter removal, heatmaps associated with radar 503 contain only reflections from human subjects which tend to be moving in an environment associated with the human subjects (e.g., user 112).

Adaptive time-domain filters, such as Kalman filters , are used to remove random body motions.

Band-pass filtering is used to separate heartbeat and respiration components from sensor data generated by radar 503.

Time frequency analysis is performed on the sensor data using a wavelet transform and a short-time Fourier transform to produce a spectrogram.

Machine learning algorithms process the spectrogram to predict the heart rate and respiratory rate from the sensor data. In some embodiments, the machine learning algorithms include any combination of a neural network, a linear regression, a support vector machine, and any other machine learning algorithm(s).

The structure described above can be extended to detect other kinds of motion associated with user 112, such as shaking.

In some embodiments, visual sensor 505 includes a depth sensor and/or an RGB sensor. Visual sensor 505 is configured to capture visual data associated with user 112. In some embodiments, this visual data includes data associated with daily activities (also referred to as activities of daily life, or ADL) performed by user 112. These daily activities may include walking, lying down, sitting down into a chair, getting out of the chair, eating, sleeping, and so on. In particular embodiments, this visual data generated by visual sensor 505, output as sensor data from visual sensor 505, is processed by video processing 506 to extract ADL features associated with daily activities described above, and features such as a sleep quality, a meal quality, a daily calorie burn rate estimation, a frequency of coughs, a visual sign of breathing difficulty, and so on. In some embodiments, video processing 506 uses machine learning algorithms such as a combination of a neural network, a linear regression, a support vector machine, and other machine learning algorithms.

Some embodiments of video processing 506 use a temporal spatial convolutional neural network, which takes a feature from a frame at a current time instant, and copies part of the feature to a next time frame. At each time frame, the temporal spatial convolutional neural network (also known as a “model”) will predict a type of activity, e.g. sitting, walking, falling, or no activity. Since an associated model generated by video processing 506 copies one or more portions of features from a current timestamp to a next timestamp, video processing 506 learns a temporal representation aggregated from a period of time to predict an associated activity.

In some embodiments, audio sensor 507 is a microphone configured to capture audio data associated with user 112. In some embodiments, audio processing 508 processes sensor data generated by audio sensor 507 using the following steps:

A time-frequency analysis performed on the sensor data generated by audio sensor 507 to generate a Mel-frequency cepstrum (MFC).

The MFC is input to a machine learning model that is configured to detect if the sensor data generated by audio sensor 507 (also known as “audio data”, “audio signal,” or “audio clip”) includes sounds associated with a cough, a wheeze, a sneeze, a snore, or another stored sound. In some embodiments, audio processing 508 uses machine learning algorithms such as a combination of a neural network, a linear regression, a support vector machine, and other machine learning algorithms.

In embodiments an output from audio processing 508 contains data that allows signal processing module 104 to determine the following conditions associated with user 112:

COPD, asthma and/or CHF associated with a cough or a wheeze.

Sleep apnea associated with snoring.

In some embodiments, training machine learning algorithms for audio processing 508 is done by using one or more datasets. These datasets include publicly-available datasets such as datasets provided from research papers, open-sourced projects with labeled datasets, videos or audio signals retrieved from a public domain with relevant labels, and so on. Datasets may also be generated in a laboratory environment using experimental data. Information retrieval techniques are used to filter out irrelevant or unreliable labels.

In some embodiments, audio processing 508 uses open-sourced and publicly available signal processing toolkits to augment an associated audio dataset into more complicated scenes. Such an augmentation involves including an audio channel associated with audio sensor 507 along with parameters such as a sample rate conversion, a volume normalization, a speed perturbation, a tempo perturbation, a background noise perturbation, a foreground audio volume perturbation, etc. In addition to augmentation, audio processing 508 also segments and clips audio signals generated by audio sensor 507 into smaller segments by removing any low-thresholding audio segments.

In some embodiments, an audio signal generated by audio sensor 507 is buffered at a 1 second interval, and snoozed every 30 milliseconds. Audio processing 508 subsequently computes Mel-frequency cepstral coefficients (MFCC) for the audio signal, which are used as features for speech recognition systems. These features are subsequently passed through a feed-forward neural network with two convolutional layers and two fully connected layers. A final prediction is thresholded to produce a final prediction. A choice of such thresholds is based on empirical evaluations.

In some embodiments, activities such as a user drinking water, laughter, footsteps, and so on may be determined by audio processing 508. In particular embodiments, a cough detection is refined to include a finer granularity level, to include dry coughing, coughing with phlegm (expectoration), and so on.

Some embodiments of audio processing 508 include more intricate neural network models, such as sequence models, with power consumption, and classification speed limit being variables corresponding to an associated design space.

The system can also be adapted to indoor and outdoor environments using appropriate datasets. This scenario can also be extended to situations with different ambient noise levels, and situations where user 112 is at variable distances from remote health monitoring system 102. The latter situation results in different signal-to-noise ratios associated with an audio signal generated by audio sensor 507. Another enhancement that can be introduced is voice recognition, where remote health monitoring system 102 is configured to recognize user 112 based on remote health monitoring system 102 learning a voice or a set of characteristic sounds associated with user 112. This offers an advantage of remote health monitoring system 102 being able to distinguish user 112 in a multi-speaker situation, where there exist multiple people in an environment, with user 112 being one of them.

In some embodiments, one or more outputs generated by detection layer 502 are received by a signal layer 510, via a communicative coupling 540. In some embodiments, signal layer 510 is configured to quantify data generated by detection layer 502. In particular embodiments, signal layer 510 generates one or more time series in response to the quantification. Signal layer 510 includes a heartbeat quantifier 512, a respiration quantifier 514, a daily activities classifier 516, a cough classifier 518, a snore classifier 520, and a wheeze classifier 522. Coupling 540 is configured such that an output from each of RF signal processing 504, video processing 506, and audio processing 508 is received by each of heartbeat quantifier 512, respiration quantifier 514, daily activities classifier 516, cough classifier 518, snore classifier 520, and wheeze classifier 522. A function of signal layer 510 is to quantify, or produce values, for outputs generated by detection layer 502. The quantifiers shown in FIG. 5 are only representative examples, and other embodiments may include additional quantifiers (such as a sneeze quantifier), or different quantifiers, or fewer quantifiers, and so forth.

In some embodiments, heartbeat quantifier 512 is configured to receive inputs from each of RF signal processing 504, video processing 506, and audio processing 508, and assign a numerical value to a heartbeat of user 112. In other words, heartbeat quantifier generates, for example, a heart rate associated with user 112.

In some embodiments, respiration quantifier 514 is configured to receive inputs from each of RF signal processing 504, video processing 506, and audio processing 508, and assign a numerical value to a respiration process associated with user 112. For example, respiration quantifier 514 may generate a respiration rate associated with user 112.

In some embodiments, daily activities classifier 516 is configured to receive inputs from each of RF signal processing 504, video processing 506, and audio processing 508, and classify one or more daily activities being performed by user 112.

A cough classifier 518 included in some embodiments of signal layer 510 is configured to characterize a cough associated with user 112, responsive to cough classifier 518 receiving inputs from each of RF signal processing 504, video processing 506, and audio processing 508. In some embodiments, cough classifier 518 is configured to characterize a cough associated with user 112. For example, user 112 may have a dry cough, or a cough with expectoration.

In some embodiments, signal layer 510 includes a snore classifier 520 that is configured to determine whether user 112 is snoring while asleep. Snore classifier 520 is useful in predicting whether user 112 has, for example, sleep apnea. Some embodiments of signal layer 510 include a wheeze classifier 522 that is configured to determine whether user 112 has a wheeze while breathing. Determining a wheeze is useful in detecting, for example, asthma, COPD, pneumonia, or other respiratory conditions associated with user 112.

In some embodiments, outputs generated by signal layer 510 are received by a fusion layer 524, via a communicative coupling 542. Fusion layer 524 is configured to process signals received from signal layer 510, in implementations using machine learning algorithms, to select and combine appropriate signals that allow fusion layer 524 to predict a severity of one or more diseases or health conditions. Fusion layer 524 includes a COPD severity classifier 526, an apnea severity classifier 258, and an asthma severity classifier 530. In some embodiments, each of COPD severity classifier 526, apnea severity classifier 528, and asthma severity classifier 530 is configured to receive an output of each of heartbeat quantifier 512, respiration quantifier 514, daily activities classifier 516, cough classifier 518, snore classifier 520, and wheeze classifier 522, via coupling 542. Fusion layer 524 essentially performs, among other functions, a sensor fusion function, where data from multiple sensors comprising sensor layer 501 are collectively processed to determine a severity of one or more health conditions associated with user 112.

In some embodiments, COPD severity classifier 526 is configured to process outputs from each of heartbeat quantifier 512, respiration quantifier 514, daily activities classifier 516, cough classifier 518, snore classifier 520, and wheeze classifier 522 to determine a severity of COPD associated with user 112. In some embodiments, apnea severity classifier 528 is configured to process outputs from each of heartbeat quantifier 512, respiration quantifier 514, daily activities classifier 516, cough classifier 518, snore classifier 520, and wheeze classifier 522 to determine a severity of OSA associated with user 112. In some embodiments, asthma severity classifier 530 is configured to process outputs from each of heartbeat quantifier 512, respiration quantifier 514, daily activities classifier 516, cough classifier 518, snore classifier 520, and wheeze classifier 522 to determine a severity of asthma associated with user 112.

Fusion layer 524 may include other classifiers, to determine a severity of any other health condition, and the classifiers 526, 528 and 530 are only given as representative examples.

In some embodiments, outputs generated by components of fusion layer 524 are received by an application layer 532 that is configured to generate a diagnosis of one or more health conditions associated with user 112. This diagnosis is generated responsive to one or more data models received from fusion layer 524 by application layer 532. In some embodiments, application layer 532 includes an AECOPD diagnosis 534 that is configured to receive an output generated by COPD severity classifier 526. In particular embodiments, AECOPD diagnosis classifier 534 is configured to determine a diagnosis of COPD associated with user 112, responsive to processing the output generated by COPD severity classifier 526. In some embodiments, application layer 532 includes an OSA diagnosis 536 that is configured to receive an output generated by apnea severity classifier 528. In particular embodiments, OSA diagnosis 536 is configured to determine a diagnosis of OSA associated with user 112, responsive to processing the output generated by apnea severity classifier 528. In some embodiments, application layer 532 includes an AAE diagnosis 538 that is configured to receive an output generated by asthma severity classifier 530. In particular embodiments, AAE diagnosis 538 is configured to determine a diagnosis of an airway adverse event (AAE) associated with user 112, responsive to processing an output generated by asthma severity classifier 530. In some embodiments, an AAE can be a manifestation of an asthma attack associated with user 112.

In some embodiments, system architecture 500 is configured to fuse, or blend data from multiple sensors such as sensor 1 106 through sensor N 110 (shown as radar 503, visual sensor 505, and audio sensor 507 in FIG. 5), and generate a diagnosis of one or more health conditions associated with user 112. In some embodiments, outputs generated by sensor 1 106 through sensor N 110 are processed by remote health monitoring system 102 in real-time to provide real-time alerts associated with a health condition such as a stoppage in breathing or a fall. In other embodiments, remote health monitoring system 102 uses historical data and historical statistics associated with user 112 to generate a diagnosis of one or more health conditions associated with user 112. In still other embodiments, remote health monitoring system 102 is configured to use a combination of real-time data generated by sensor 1 106 through sensor N 110 along with historical data and historical statistics associated with user 112 to generate a diagnosis of one or more health conditions associated with user 112.

Using a sensor fusion approach allows for a greater confidence level in detecting and diagnosing a health condition associated with user 112. Using a single sensor is prone to increasing a probability associated with incorrect predictions, especially when there is an occlusion, a blindspot, a long range or multiple people in a scene as viewed by the sensor. Using multiple sensors in combination, and combining data processing results from processing discrete sets of quantitative data generated by the various sensors, produces a more accurate prediction, as different sensing modalities complement each other in their capabilities. Examples of how outputs from multiple sensors with distinct sensing modalities may be used to determine one or more health conditions are provided below.

Outputs from radar 503 and visual sensor 505 can be used to determine a heart rate and a respiratory rate associated with user 112, where radar 503 is configured to detect fine motions associated with user 112, and visual sensor 505 (a depth sensor or an RGB sensor) is used to capture visual data associated with movements of user 112 and a physical position of user 112 (e.g., laying down in bed). Data generated by visual sensor 505 can also be processed to predict a heart rate and a respiratory rate. These results can be combined with results from processing data generated by radar 503 to generate a more accurate diagnosis.

A combination of data generated by audio sensor 507 and visual sensor 505 is used to detect a cough in user 112. In this case, results from processing audio data from audio sensor 507 are combined with results from processing visual data from visual sensor 505 to determine a presence and a nature of a cough associated with user 112, at a higher confidence level than if data from either sensor was used singularly.

Visual sensor 505 is useful in an environment that includes multiple users, where one or more vital signs of a specific user of the multiple users need to be continuously tracked. For example, data from visual sensor 505 can be processed by signal processing module 104 to determine a difference between two or more individuals in an environment based on their height, body shape, facial features, and motion characteristics (e.g., gait, posture, and so on). In some embodiments, this tracking process is accomplished using visual sensor 505 in conjunction with radar 503 and audio sensor 507.

Remote health monitoring system 102 can also be configured to perform the following functions:

Using radar 503 for any combination of fall detection, and position and speed detection of user 112.

Using visual sensor 505 for fall detection.

Using audio sensor 507 to detect coughing, wheezing, sneezing, or snoring.

Predicting an acute exacerbation of COPD using features derived from heart rate, respiratory rate, and coughing. Some examples of derived features include detected anomalies of heart rate and respiratory rate (e.g., abnormal beats per minute (bpm) compared to a same time of the day historically, acute changes of bpm in a short period of time), a frequency of coughing, a frequency of productive coughing, etc. Remote health monitoring system 102 can also detect body motions associated with a cough and give an estimation of how dangerous the cough is in terms of body balance, gait and other body metrics.

Determining CHF exacerbation by predicting based on derived features like a high heart rate at night, a high lung fluid index, a specific activity level (derived from activity detection), and so on.

Predicting asthma exacerbating based on features (or derived features) such as a respiratory rate, wheezing, a heart rate, an activity level, and so on.

Other embodiments of remote health monitoring system 102 include combining signals and predictions from vision and radar signals to improve a prediction accuracy. This approach is based on combinations of predictions from multiple sensors and/or models providing a prior knowledge or a secondary opinion to an audio prediction model. This, in turn, allows a process where arbitrary models can be ensembled into a unified prediction framework. Such a model ensemble framework may rely on feedforward neural networks, bootstrapping aggregating, boost, Bayesian parameter averaging framework or Bayesian model combination.

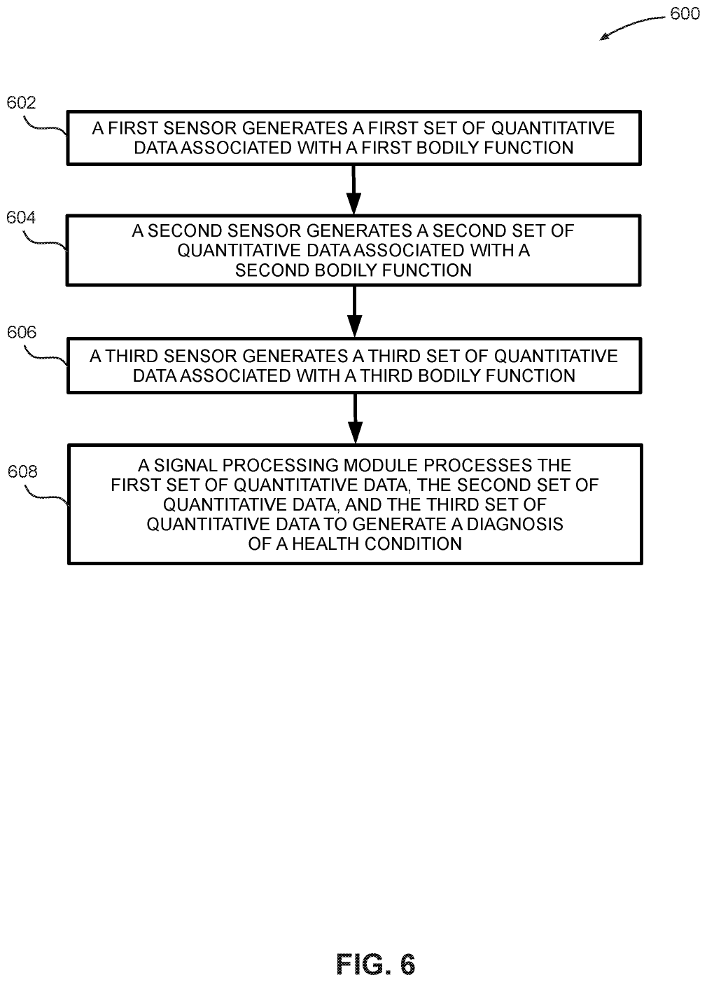

FIG. 6 is a flow diagram depicting an embodiment of a method 600 to generate a diagnosis of a health condition. At 602, a first sensor generates a first set of quantitative data associated with a first bodily function. In some embodiments, the first sensor is radar 503, the first set of quantitative data is associated with one or more RF signals received by radar 503, and the first bodily function is a heartbeat or a respiration. At 604, a second sensor generates a second set of quantitative data associated with a second bodily function. In some embodiments, the second sensor is visual sensor 505, the second set of quantitative data is associated with one or more visual signals received by visual sensor 505, and the second bodily function is an ADL. At 606, a third sensor generates a third set of quantitative data associated with a third bodily function. In some embodiments, the third sensor is audio sensor 507, the third set of quantitative data is associated with one or more audio signals received by audio sensor 507, and the third bodily function is a cough, a snore or a wheeze. At 608, a signal processing module processes the first set of quantitative data, the second set of quantitative data, and the third set of quantitative data to generate a diagnosis of a health condition. In some embodiments the signal processing module is signal processing module 104 that is configured to implement detection layer 502, signal layer 510, fusion layer 524, and application layer 532, and generate any combination of outputs from AAE diagnosis 538, OSA diagnosis 536, and AECOPD diagnosis 534. In implementations, however, any of the layers may have different, more, or fewer elements to diagnose different, or more, or fewer health conditions. In implementations one or more of the steps of method 600 may be performed in a different order than that presented.

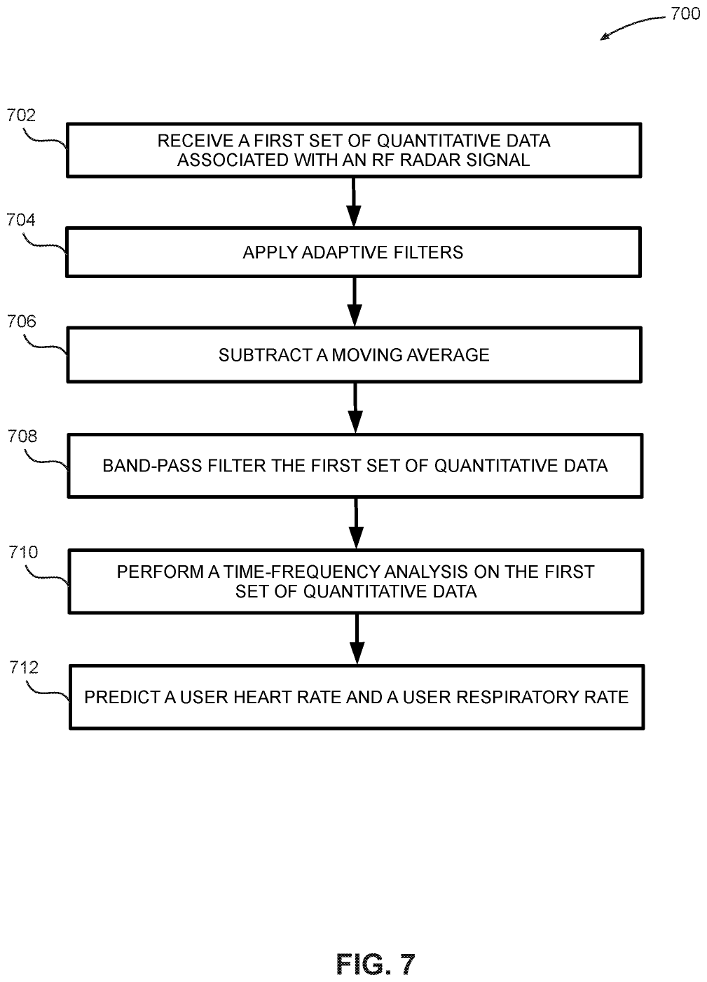

FIG. 7 is a flow diagram depicting an embodiment of a method 700 to predict a user heart rate and a user respiratory rate. At 702, the method receives a first set of quantitative data associated with an RF radar signal. In some embodiments, the RF radar signal is associated with radar 503. In particular embodiments, the first set of quantitative data is associated with a bodily function such as a heartbeat or a respiration associated with, for example, user 112. At 704, the method applies adaptive filters to eliminate random body motion associated with user 112. At 706, the method performs static clutter removal on the received data by subtracting a moving average. At 708, the method performs band pass filtering on the first set of quantitative data to separate out heartbeat and respiration components associated with the first set of quantitative data. At 710, the method performs a time-frequency analysis on the first set of quantitative data using a wavelet transform, to produce a spectrogram. In particular embodiments, a short-time Fourier transform is used in conjunction with the wavelet transform to produce the spectrogram. At 712, the method processes the spectrogram, in implementations using deep learning models (i.e., machine learning models such as deep convolutional networks), to predict a heart rate and a respiratory rate associated with, for example, user 112. In some embodiments, steps 702 through 712 are performed by signal processing module 104. In implementations one or more of the steps of method 700 may be performed in a different order than that presented.

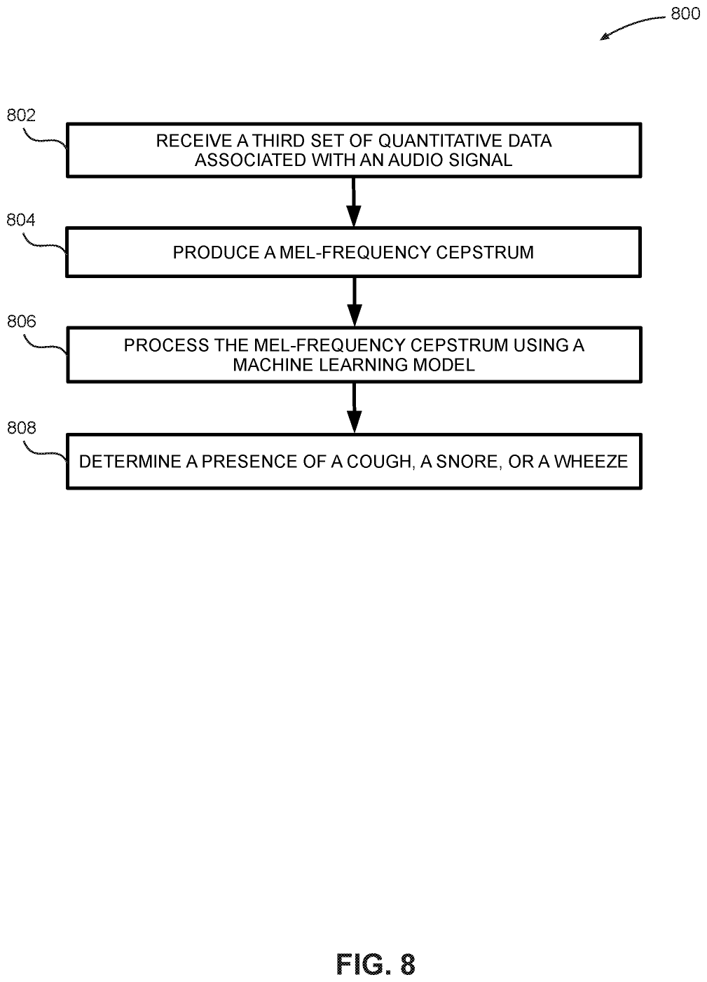

FIG. 8 is a flow diagram depicting an embodiment of a method 800 to determine a presence of a cough, a snore, or a wheeze. At 802, the method receives a third set of quantitative data associated with an audio signal. In some embodiments, the audio signal is generated by audio sensor 507. At 804, the method processes the audio data and generates a Mel-freqency cepstrum (MFC). Next, at 806, the method processes the Mel-frequency cepstrum, in implementations using a machine learning model. In some embodiments, the machine learning model is a combination of a neural network, a linear regression, a support vector machine, and other machine learning algorithms. At 808, the method determines a presence of a cough, a snore, or a wheeze, in implementations based on an output of the machine learning model. In some embodiments, steps 802 through 808 are performed by signal processing module 104.

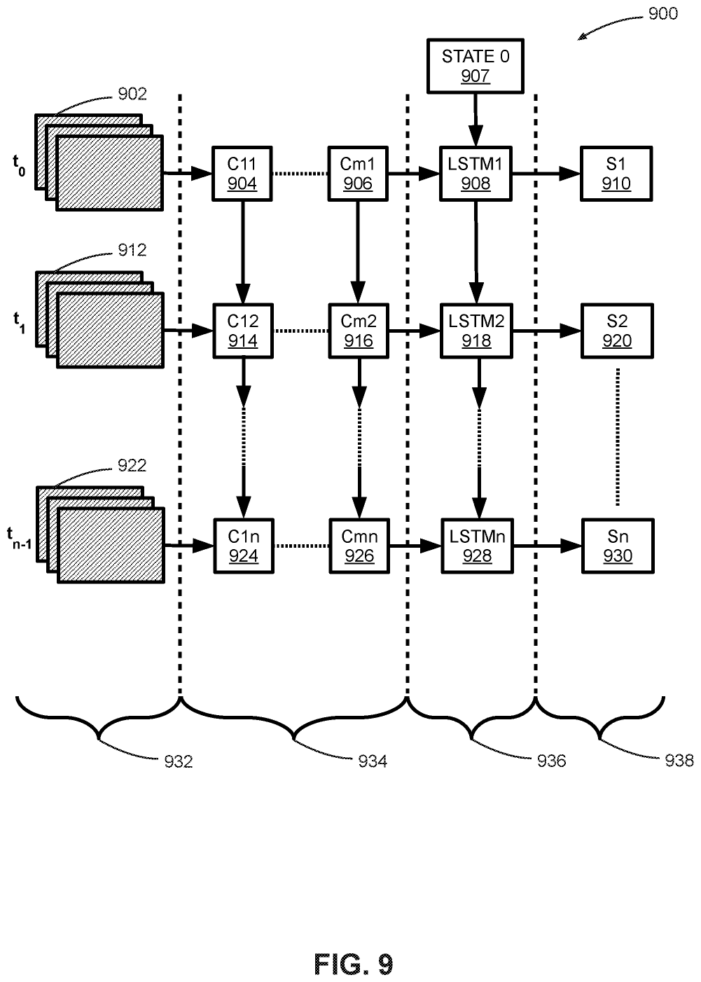

FIG. 9 is a schematic diagram depicting a processing flow 900 of multiple heatmaps using neural networks. In some embodiments, processing flow 900 is configured to function as a fall classifier that determines whether user 112 has had a fall. In some embodiments, processing flow 900 processes a temporal set of heatmaps 932 that includes a first set of heatmaps 902 at a time t₀, a second set of heatmaps 912 at a time t₁, through an n^{th} set of heatmaps at a time tₙ₋₁. In implementations, receiving temporal set of heatmaps 932 comprises a preprocessing phase for processing flow 900.

In some embodiments, time t₀, time t₁through time tₙ₋₁ are consecutive time steps, with a fixed-length sliding window (e.g., 5 seconds). Temporal set of heatmaps 932 is processed by a multi-layered convolutional neural network 934. Specifically, first set of heatmaps 902 is processed by a first convolutional layer C11 904 and so on, through an m^{th} convolutional layer Cm1 906; second set of heatmaps 912 is processed by a first convolutional layer C12 914 and so on, through an m^{th} convolutional layer Cm2 916; and so on through n^{th} set of heatmaps 922 being processed by a first convolutional layer C1n 924, through an m^{th} convolutional layer Cmn 926. In some embodiments, a convolutional layer with generalized indices Cij is configured to receive an input from a convolutional layer C(i−1)j for i>1, and a convolutional layer Cij is configured to receive an input from convolutional layer Ci(j−1) for j>1. For example, convolutional layer Cm2 916 is configured to receive an input from a convolutional layer C(m−1)2 (not shown in FIG. 9), and from convolutional layer Cm1 906.

Collectively, first convolutional layer C11 904 through m^{th} convolutional layer Cm1 906, first convolutional layer C12 914, through m^{th} convolutional layer Cm2 916 and so on, through first convolutional layer C1n 924, through m^{th} convolutional layer Cmn 926 comprise multi-layered convolutional neural network 934 that is configured to extract salient features at each timestep, for each of the first set of heatmaps 902 through the n^{th} set of heatmaps 922.

In some embodiments, outputs generated by multi-layered convolutional neural network 934 are received by a recurrent neural network 936 that is comprised of a long short-term memory LSTM1 908, a long short-term memory LSTM2 918, through a long short-term memory LSTMn 928. In some embodiments, long short-term memory LSTM1 908 is configured to receive an output from m^{th} convolutional layer Cm1 906 and an initial system state 0 907, long short-term memory LSTM2 918 is configured to receive inputs from long short-term memory LSTM1 908 and m^{th} convolutional layer Cm2 916 and so on, through long short-term memory LSTMn 928 being configured to receive inputs from a long short-term memory LSTM(n−1) (not shown but implied in FIG. 9) and m^{th} convolutional layer Cmn 926. Recurrent neural network 936 is configured to capture complex spatio-temporal dynamics associated with temporal set of heatmaps 932 while taking into account the multiple discrete time steps t₀ through tₙ₋₁.

In some embodiments, an output generated by each of long short-term memory LSTM1 908, long short-term memory LSTM2 918, through long short-term memory LSTMn 928 is received by a softmax S1 910, a softmax S2 920, and so on through a softmax Sn 930, respectively. Collectively, softmax 51 910, softmax S2 920 through softmax Sn 930 comprise a classifier 938 that is configured to categorize an output generated by the corresponding recurrent neural network to determine whether user 112 has had a fall at a particular time instant in a range of t₀ through tₙ.

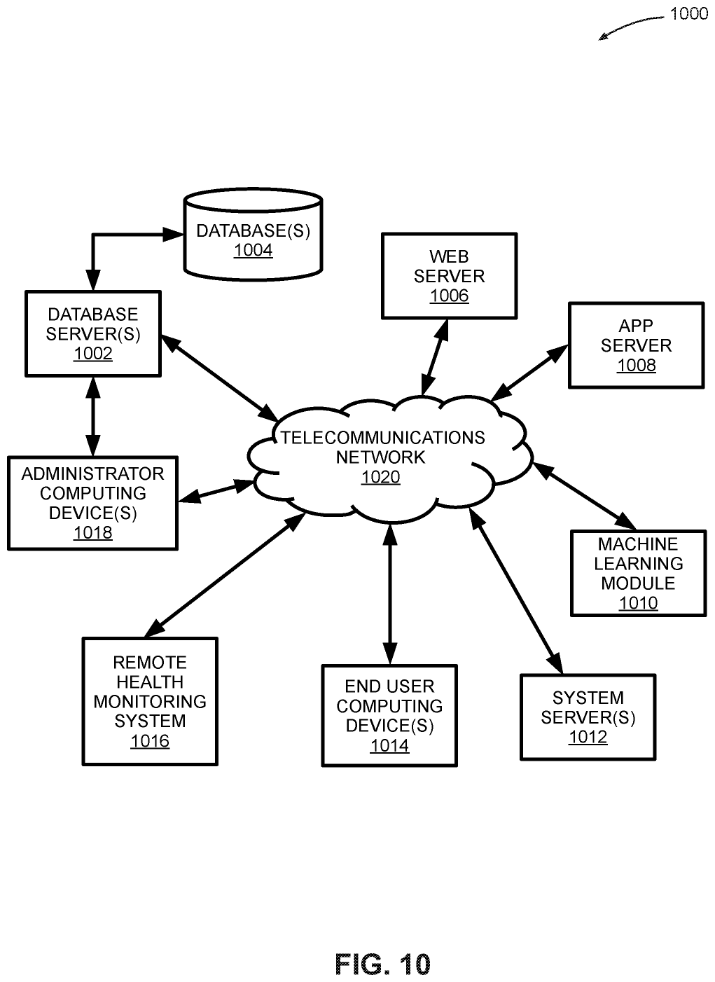

FIG. 10 is a block diagram depicting an embodiment of a system architecture 1000 of a remote health monitoring system. In some embodiments, architecture 1000 includes a remote health monitoring system 1016 that includes the functionalities, subsystems and methods described herein. Remote health monitoring system is coupled to a telecommunications network 1020 that can include a public network (e.g., the Internet), a local area network (LAN) (wired and/or wireless), a cellular network, a WiFi network, and/or some other telecommunication network.

Remote health monitoring system 1016 is configured to interface with an end user computing device(s) 1014 via telecommunications network 1020. In some embodiments, end user computing device(s) can be any combination of computing devices such as desktop computers, laptop computers, mobile phones, tablets, and so on. For example, an alarm generated by remote health monitoring system 1016 may be transmitted by remote health monitoring system 1016 to an end user computing device in a hospital to alert associated medical personnel of an emergency (e.g., a fall).

In some embodiments, remote health monitoring system 1016 is configured to communicate with a system server(s) 1012 via telecommunications network 1020. System server(s) 1012 is configured to facilitate operations associated with system architecture 1000, for example signal processing module 104 may be implemented using a server communicatively coupled with sensors.

In some embodiments, remote health monitoring system 1016 communicates with a machine learning module 1010 via telecommunications network 1020. Machine learning module 1010 is configured to implement one or more of the machine learning algorithms described herein, to augment a computing capability associated with remote health monitoring system 1016. Machine learning module 1010 could be located on one or more of the system server(s) 1012.

In some embodiments, remote health monitoring system 1016 is enabled to communicate with an app server 1008 via telecommunications network 1020. App server 1008 is configured to host and run one or more mobile applications associated with remote health monitoring system 1016.

In some embodiments, remote health monitoring system 1016 is configured to communicate with a web server 1006 via telecommunications network 1020. Web server 1006 is configured to host one or more web pages that may be accessed by remote health monitoring system 1016 or any other components associated with system architecture 1000. In particular embodiments, web server 1006 may be configured to serve web pages in a form of user manuals or user guides if requested by remote health monitoring system 1016, may allow administrators to monitor operation and/or data collection of the remote health monitoring system 100, adjust system settings, and so forth remotely or locally.

In some embodiments a database server(s) 1002 coupled to a database(s) 1004 is configured to read and write data to database(s) 1004. This data may include, for example, data associated with user 112 as generated by remote health monitoring system 102.

In some embodiments, an administrator computing device(s) 1018 is coupled to telecommunications network 1020 and to database server(s) 1002. Administrator computing devices(s) 1018 in implementations is configured to monitor and manage database server(s) 1002, and monitor and manage database 1004 via database server(s) 1002. It may also allow an administrator to monitor operation and/or data collection of the remote health monitoring system 100, adjust system settings, and so forth remotely or locally.

Although the present disclosure is described in terms of certain example embodiments, other embodiments will be apparent to those of ordinary skill in the art, given the benefit of this disclosure, including embodiments that do not provide all of the benefits and features set forth herein, which are also within the scope of this disclosure. It is to be understood that other embodiments may be utilized, without departing from the scope of the present disclosure.

What is claimed is:

1. An apparatus configured to perform a contact-free detection of one or more health conditions, the apparatus comprising:
a plurality of sensors configured for contact-free monitoring of at least one bodily function; and
a signal processing module communicatively coupled with the plurality of sensors;
wherein the signal processing module is configured to receive data from the plurality of sensors;
wherein a first sensor of the plurality of sensors is configured to generate a first set of quantitative data associated with a first bodily function;
wherein a second sensor of the plurality of sensors is configured to generate a second set of quantitative data associated with a second bodily function;
wherein a third sensor of the plurality of sensors is configured to generate a third set of quantitative data associated with a third bodily function;
wherein the signal processing module is configured to process the first set of quantitative data, the second set of quantitative data, and the third set of quantitative data, and wherein the signal processing module is configured to process at least one of the sets of quantitative data using a machine learning module; and
wherein the signal processing module is configured to generate, responsive to the processing, at least one diagnosis of a health condition.

2. The apparatus of claim 1, wherein the first bodily function is one of heartbeat and respiration, wherein the second bodily function is a daily activity, and wherein the third bodily function is one of coughing, snoring, expectoration and wheezing.

3. The apparatus of claim 1, wherein the first sensor is a radar, wherein the second sensor is a visual sensor, and wherein the third sensor is an audio sensor.

4. The apparatus of claim 3, wherein the radar is a millimeter wave radar, wherein the visual sensor is one of a depth sensor and an RGB sensor, and wherein the audio sensor is a microphone.

5. The apparatus of claim 3, wherein the radar is configured to generate quantitative data associated with one of heartbeat and breathing, wherein the visual sensor is configured to generate quantitative data associated with a daily activity, and wherein the audio sensor is configured to generate quantitative data associated with one of coughing, snoring, wheezing and expectoration.

6. The apparatus of claim 3, wherein data generated using the audio sensor is processed using a combination of a Mel-frequency Cepstrum and a deep learning model associated with the machine learning module.

7. The apparatus of claim 3, wherein data generated using the radar is processed using one of static clutter removal, band pass filtering, time-frequency analysis, wavelet transforms, spectrograms, and a deep learning model associated with the machine learning module.

8. The apparatus of claim 1, wherein the health condition is a respiratory health condition.

9. The apparatus of claim 8, wherein the respiratory health condition is one of OSA, COPD, and asthma.

10. The apparatus of claim 1, wherein results from processing the first set of quantitative data, the second set of quantitative data, and the third set of quantitative data are combined to generate the diagnosis.

11. A method for performing a contact-free detection of one or more health conditions, the method comprising:
generating, using a first sensor of a plurality of sensors, a first set of quantitative data associated with a first bodily function of a body, wherein the first sensor does not contact the body;
generating, using a second sensor of the plurality of sensors, a second set of quantitative data associated with a second bodily function of the body, wherein the second sensor does not contact the body;
generating, using a third sensor of the plurality of sensors, a third set of quantitative data associated with a third bodily function of the body, wherein the third sensor does not contact the body;
processing, using a signal processing module, the first set of quantitative data, the second set of quantitative data, and the third set of quantitative data, wherein the signal processing module is communicatively coupled with the plurality of sensors, and wherein at least one of the first set of quantitative data, the second set of quantitative data, and the third set of quantitative data is processed using a machine learning module; and
generating, using the signal processing module, responsive to the processing, at least one diagnosis of a health condition.

12. The method of claim 11, wherein the first bodily function is one of heartbeat and respiration, wherein the second bodily function is a daily activity, and wherein the third bodily function is one of coughing, snoring, sneezing, expectoration and wheezing.

13. The method of claim 11, wherein the first sensor is a radar, wherein the second sensor is a visual sensor, and wherein the third sensor is an audio sensor.

14. The method of claim 13, wherein the radar is a millimeter wave radar, wherein the visual sensor is one of a depth sensor and an RGB sensor, and wherein the audio sensor is a microphone.

15. The method of claim 13, further comprising:
generating, using the radar, quantitative data associated with one of heartbeat and respiration;
generating, using the visual sensor, quantitative data associated with a daily activity; and
generating, using the audio sensor, quantitative data associated with one of coughing, snoring, sneezing, wheezing and expectoration.

16. The method of claim 13, further comprising:
receiving, by the signal processing module, the first set of quantitative data associated with an RF signal generated using the radar;
subtracting, using the signal processing module, a moving average associated with the first set of quantitative data;
band-pass filtering, using the signal processing module, the first set of quantitative data;
performing, using the signal processing module, time-frequency analysis on the first set of quantitative data using wavelet transforms; and
predicting, using the signal processing module, a user heart rate and a user respiratory rate using a deep learning model and a spectrogram function.

17. The method of claim 13, further comprising:
receiving, using the signal processing module, the third set of quantitative data associated with an audio signal from the audio sensor;
producing, using the signal processing module, a Mel-frequency cepstrum using time-frequency analysis performed on the third set of quantitative data; and
determining, using the signal processing module, a presence of one of a cough, a snore and a wheeze associated with a user.

18. The method of claim 11, wherein the health condition is a respiratory health condition.

19. The method of claim 18, wherein the respiratory health condition is one of OSA, COPD, and asthma.

20. The method of claim 11, wherein results from processing the first set of quantitative data, the second set of quantitative data, and the third set of quantitative data are combined to generate the diagnosis.
